(19) United States
(12) Patent Application Publication
(10) Pub. No.: US 2023/0303963 A1
(43) Pub. Date: Sep. 28, 2023

(54) MICROBIOLOGICAL METHOD AND EQUIPMENT FOR THE FRACTIONATING AND UTILIZATION OF SLAUGHTER HOUSE AND SAUCE INDUSTRY WASTES AND SIDE STREAMS

(71) Applicant: Eino Elias HAKALEHTO, Kuopio (FI)

(72) Inventor: Eino Elias HAKALEHTO, Kuopio (FI)

(21) Appl. No.: 18/021,558

(22) PCT Filed: Aug. 17, 2021
(86) PCT No.: PCT/FI2021/000007

§ 371 Date: Feb. 16, 2023

(30) Foreign Application Priority Data
Aug. 17, 2020 (FI) 20200055

(51) Int. Cl. C12N 1/20; C05F 1/00; C05F 17/20; C05F 17/40; C05G 5/20; C11D 9/00; B09B 3/60

(52) U.S. Cl. CPC C12N 1/20; C05F 1/005; C05F 17/20; C05F 17/40; C05G 5/20; C11D 9/005; B09B 3/60; C12R 2001/44

(57) ABSTRACT

Various waste fractions from meat, gravy, and animal feed industries, that are difficult to recycle, as well as wastes, for example, from fishing and fish processing, are often challenging to separate for further processing into products. The hygienic and otherwise safe and economical utilization of these, consistent with ecologically sustainable development, requires the development of new methods. According to the method and apparatus of this invention, with the help of microbes and their enzymes, for example, bone and tissue waste, as well as protein, fat material or materials containing blood matte, that have been separated during meat processing, can be fractioned into new raw materials. Gained products can be, for example, energy fractions and gases, waxes and various organic fertilizers and soil improvement substances.

### BACKGROUND

Fractionation of different waste materials can be more difficult when dealing with side streams. Many types of mixed waste would be beneficial to recycle to retrieve and utilize the materials. Thermal treatments, extractions and other methods of waste handling can cause unwanted sedimentation. Microbes and their enzymes offer a useful and efficient solution for recycling of the side streams (Hakalehto & Jääskeläinen 2017). In this case, problems might arise from reactions between different substances. Genetical methods used to manipulate microbial cultures with several beneficial features in waste handling might cause severe environmental threats. This makes it desirable to recover natural microbial cultures that naturally possess the features in them.

We have often utilized mixed cultures to combine their useful features. These types of solutions have been used i.e., industrial waste (Den Boer et al., 2016; Schwede et al., 2017). This requires advanced development and metabolic studies in many parallel processes. Besides above-mentioned waste and side streams, other types of applicable biomass can be added into the process as well, if suitable combination of microbial cultures are available.

When leading the processes into microbiologically and biochemically desirable direction the conditions are made as suitable for relevant cultures as possible so that they reach adequately strong presence in the mixed culture. Discovering and testing suitable selective features is especially important in developing the process. Certain physicochemical features of the process, or their combinations, such as the temperature. pH, osmolarity, oxygen concentration, etc., can be used to select the desired cultures.

Processing slaughterhouses waste can yield many products (Hakalchto et al. 2016 a, b). Residues from this process can be used to produce meat bone meal, which is excellent organic fertilizer and soil conditioner (Kivelä and Hakalehto, 2016). Microbiological and biochemical safety by hygienization is important in certain processes involving industrial waste or severe issues might arise (Hakalehto 2015a; Armon, 2015; Hakalehto et al., 2015a; Hakalehto & Heitto, 2015; Pesola et al. 2015). Antibiotic resistant, bacteria evolving in industrial waste which might be released into the environment, or the product flows of the circulation economy is another problem (Hakalchto, 2015a). Microbiological and toxicological risk management in meet processing requires continuous monitoring of the side streams, intermediaries, and products (Hakalchto et al., 2015a).

### DESCRIPTION

Since waste from abattoirs is commonly used to produce food for domestic pets, fur farm animals, and to produce raw materials for gravies, it is necessary to hygienize them. Production of soil improvements and fertilizers must correspondingly pay attention to safety issues. Heat or drought and other simple methods can be applied to these processes. This can, however, cause thickening and gelatinization of the materials. Especially waste originating from bone tissue can yield soft tissue and bone marrow, which requires processing before the bone can be pulverized.

When the above-mentioned sauce industry waste or other waste from meat processing of slaughtered animals is used for direct utilization, refinement, fractionation, and purification purposes, by applying microbial strains, it is advantageous to find for these purposes microbes, which possess as many useful purposes as possible. These types of cultures can be found from Staphylococcus bacteria. Results from research projects focusing on Staphylococcus haemolyticus, for example, are available (Samgina et al., 2016). This bacterium has hemolytic, proteolytic, and lipolytic enzymes. Similar can be found from Bacillus cereus, for instance (Hakalehto and Heitto, 2015). Using both, Staphylococcus and Bacillus bacteria, and other microbes with similar features, in the method described in this application, makes it possible to reline and factorize the above-mentioned meat-based organic waste. These bacteria are Gram-positive. Animal blood can also be processed in a similar manner. Especially processing chicken blood becomes possible. This is based on dissolvement of the blood cells. Also, protein, lipid, and other blood-containing waste such as waste from fisheries can be treated this way.

Because the above-mentioned wastes and side streams contain plenty of natural microbes, whose elimination can be difficult despite different hygienization methods, it is beneficial to utilize selective methods known in microbiology. In the selection of staphylococci, salt (NaCl), with a concentration of, for example, 7.5%, is generally used in the culture. The use of salt is one of the many possibilities to effectuate selection when the bacteria in question are added to wastes or side streams. Other salts can also be experimented as selective factors. As a selective factor, also a lower, under 2.5% salt concentration can be used. See Example 1.

It is also important, to get from the bacterial strain used for inoculation, an inoculum that is potent enough. In this case, one or more inoculation fermenters, that are solidly fixed to a reactor, can be used, and with the help of these, several inoculations can be carried out, for example, every few hours.

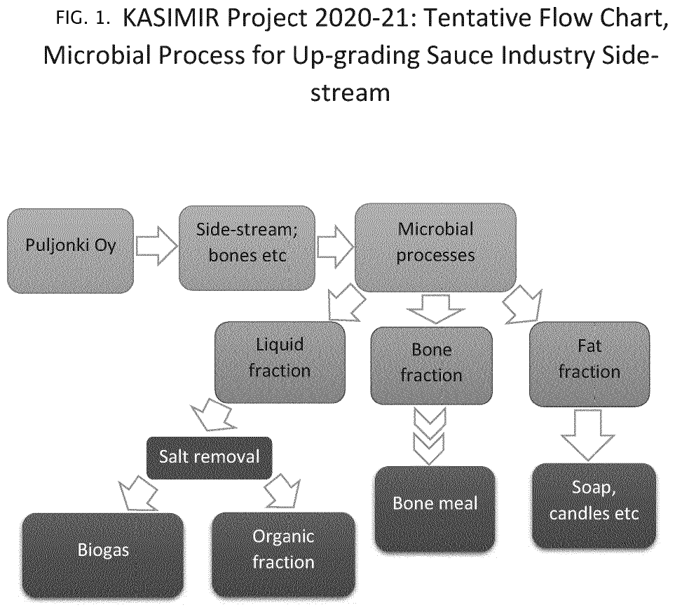

With the help of an industrial process, according to this invention, raw material for the manufacture of different products, can be gained (FIG. 1). Possible components that are thus developed are waxlike soap and candle fractions, biogas produced for energy use, or organic fertilizer fractions and bone meal produced for fertilizing use. For fertilizing use, the amount of organic nitrogen can be increased microbiologically (Hakalehto 2018). See also Example 2.

### Example 1

The microbiological treatment of waste from sauce industry was carried out in a 10 m³ sized tank, with a built-in blending system, on so called “Ape”-wagon.

1. Work Performance

1.1 Preparation of Laboratory Inocula
6 liters of TYG broth was prepared in the laboratory into 0.5 liter bottles, the bottles were autoclaved
Staphylococcus haemolyticus strain, from deep freeze storage, then grown on a ChromAgar Orientation™ dish, was inoculated into the bottles
cleanliness of all bottles was checked with plating

1.2 Preparation of the Inoculations of the Seed Fermenter
meat bonemeal was heated in the laboratory in an oven at +120 Celsius degrees (for 4 hours)
a 300 liter seed fermenter was washed with hot water
160 liters of hot tap water (approximately 50 degrees Celsius), was led into the seed fermenter, and to this was mixed
a. 3.5 liters of salt (about 2%)
b. 2.2 liters of meat bone meal
c. 0.8 liters of molasses
d. 0.6 liters of milk powder
Thereafter 20 liters of cold water was added. The pH of the seed fermenter was adjusted to the value of 6.5 and the temperature (T) was 40° C. (seed fermenter meter)
20 liters of cold water was added for cooling and the seed fermenter temperature (T) was adjusted to 40° C.
liters (4 bottles) of fully grown S. haemolyticus bacterial broth was added
the circulation water heating was first set to +44° C. and then later in the evening dropped to +36 degrees
the following morning the temperature of the seed fermenter was 35° C. and the pH 5.5
8 liters of bouillon, that had been stored outside for about 2 months, was added (side stream from sauce industry)
the inoculation was pumped into the reactor
a new growth medium was prepared into the seed fermenter, similarly to the previous day, except that the bone mass of the bouillon was left on the bottom
microbe culture (2 liters) was added
the following morning the T of the seed fermenter was 38 degrees and pH 4.5
half (about 100 liters) of the content of the seed fermenter was pumped into the reactor
the rest of the inoculation was pumped into the reactor in the afternoon
a new growth medium was prepared into the seed fermenter, similarly to the previous day, except that 0.5 liters of granular garden lime was added to slow the pH decrease
the following morning the seed fermenter pH was 4.5
the inoculation was pumped into the reactor

### The Loading and Inoculation of the Reactor

3150 kg of bouillon that had been stored outdoors and 1.5 m³ of hot water, to which 35 kg of salt (about 2.5% calculated per liquid volume) was dissolved, were loaded into the reactor
reactor's T 19 degrees
the temperature of the circulation water was increased to 60 degrees, the water circulation of the seed fermenter was shut down
in the afternoon 1′ 28 degrees, pH 7
the inoculation was pumped into the reactor
the surface of the reactor liquid and foam 105 cm from the rim
the following morning the surface was 15 cm from the rim, from where it flowed into the extraction pipe
sampling through the bottom valve, which about 10 cm from the bottom, foam pH 7
new inoculations were prepared on two days (as above)
reactor's T during the test 30-39 degrees
part of the mass was taken from the reactor after 1-2 days and part after 5-6 days to find out whether the lengthening of the reaction time changes bone composition
samples were taken from the reactor daily to monitor the microbe situation

### Results

to monitor the appearance of Staphylococcus haemolyticus strain, microbe samples were taken from the seed fermenter and reactor, and these were plated. The platings were done on ChromAgar Orientation® and ST110 plates.

| | | appearance of | | | |
|---|---|---|---|---|---|
| | | Staphylococcus | | | |
| date | sample | haemolyticus | T | pH | |
| 26 Apr. 2021 | seed fermenter | − | 40 | 6.5 | some small dark blue colonies, |
| | before inoculation | | | | later some mould colonies |
| 26 Apr. 2021 | seed fermenter | + | | | in addition, some small dark blue |
| | after inoculation | | | | colonies, later some mould |
| | | | | | colonies |
| 27 Apr. 2021 | seed fermenter | + | 35 | 5.5 | in addition lots of dark blue |
| | | | | | colonies, some Proteus and |
| | | | | | E. coli-colonies |
| 27 Apr. 2021 | reactor | + | 30 | 7 | in addition lots of dark blue |
| | | | | | colonies, some Proteus and |
| | | | | | E. coli-colonies and some moulds |
| 28 Apr. 2021 | seed fermenter | + | 38 | 4.5 | in addition lots of dark blue |
| | | | | | colonies, some Proteus and |
| | | | | | E. coli-colonies and some moulds |
| 28 Apr. 2021 | reactor | + | 39 | 7 | in addition lots of dark blue |
| | | | | | colonies, some Proteus and |
| | | | | | E. coli-colonies and some moulds |
| 30 Apr. 2021 | reactor | + | | 7 | in addition lots of dark blue |
| | | | | | colonies, some Proteus and |
| | | | | | E. coli-colonies, less moulds |
| 1 May 2021 | reactor | + | | 7.5 | in addition lots of dark blue |
| | | | | | colonies, some Proteus, E. coli |
| | | | | | and Bacillus-colonies, less |
| | | | | | moulds |
| 3 May 2021 | reactor | + | | 7 | in addition lots of dark blue |
| | | | | | colonies, some Proteus, E. coli |
| | | | | | and Bacillus-colonies, less |
| | | | | | moulds, less Staphylococcus |
| | | | | | colonies |

the Staphylococcus strain (Staphylococcus haemolyticus) that was added into the process, preserved in the reactor throughout the experiment
this bacterium and the process resulted in the waste material's bone matter to separate from other solid matter, and in the cleansing and embrittlement of the bones so that these could be further processed into bone meal. It was noted that this bone meal was superbly suitable as an organic fertilizer.

### Example 2

Based on the tests in laboratory dish, in the analysis of the bone meal, the concentration of fertilizing nitrogen was almost optimal
There is 4% of nitrogen, which is quite a lot, but also quite in line with foreign results
There is 11-12% of phosphorus, while the theoretical maximum would be 15%. In practice, only bioapatite ([Ca10(PO4)6(OH)2]) and a little of slowly dissolving protein and about 10% of water have remained in the material
a good component for organic phosphorus fertilizer can be gained from this produce, but the usability of its phosphorus needs to be investigated separately. At least in acidic soil, the bioapatite's phosphorus should be brought to plants' use.

### REFERENCES

Armon, R (2015). Food borne viruses. In: Hakalehto, F., (ed.) Microbiological Food Hygiene. New York, NY, USA: Nova. Science Publishers, Inc.
Den Boer, E., Lucaszewska, A., Kluczkicwicz, D., Lewandowska, D., King. K., Reijonen, T., Suhonen, A., Jääskeläinen, A., Heitto, A., Laatikainen, R., Hakalehto, E. (2016). Volatile fatty acids as an added value from biowaste. Waste Management, 58: 62-69.
Hakalehto, E. (2015a). Hazards and prevention of food spoilage. In: Hakalehto, E. (ed.) Microbiological Fond Hygiene. New York, NY, USA: Nova Science Publishers, Inc.
Hakalehto, E. (2015b). Antibiotic resistance in foods. In: Hakalehto. E. (ed.) Microbiological food hygiene. New York, NY, USA: Nova Science Publishers. Inc.
Hakalehto, F. (20161. The many microbiomes. In: Hakalehto, E. (ed.) Microbiological Industrial Hygiene. New York, NY, USA: Nova Science Publishers. Inc.
Hakalehto, E., Heitto, A. (2015). Detection of Bacillus cercus. In: Hakalehto, E. (ed.) Microbiological Food Hygiene. New York, NY, USA: Nova Science Publishers, Inc.
Hakalehto, E., Jääskeläinen, A. (2017). Reuse and circulation of organic resources and mixed residues. In: Dahlquist, E. and Hellstrand, S. (Eds.) Natural resources available today and in the future: how to perform change management for achieving a sustainable world. Springer Verlag, Germany.
Hakalehto, E., Pesola, J., Heitto, A., Hänninen, H., Hendolin, P., Hänninen O., Armon, R., Humppi, T., Paakkanen, H. (2015a). First detection of Salmonella contaminations. In: Hakalehto, E. (ed.) Microbiological Food Hygiene. New York, NY, USA: Nova Science Publishers, Inc.
Hakalehto, E., Heitto. A., Jokelainen, J., Heitto, L. (2015b). Monitoring food and water sources with the PMEU. In: Hakalehto, F. (ed.) Microbiological Food Hygiene. New York, NY, USA: Nova Science Publishers, Inc.
Hakalehto, F., Heitto. A., Kivelä, J., Laatikainen, R. (2016a). Meat industry hygiene, outlines of safety and material recycling by biotechnological means. In: Hakalehto, F. (ed.) Microbiological Industrial Hygiene. New York, NY, USA: Nova Science Publishers. Inc.
Hakalehto, E., Heitto, A., Andersson. H., Lindmark, J., Janson, J., Reijonen, T., Suhonen, A., Jääskeläinen, A., Laatikainen, R., Schwede, S., Klintenberg, P., Thorin, E. (2016b). Some remarks on processing of slaughterhouse wastes from ecological chicken abattoir and farm. In: Hakalehto, E. (ed.) Microbiological Industrial Hygiene. New York, NY, USA: Nova Science Publishers, Inc.
Kivelä, J., Hakalehto, E. (2016). Fertilization uses of meat bone meal and effects on microbial activity. In: Hakalehto, E. (ed.) Microbiological Industrial Hygiene. New York, NY. USA: Nova Science Publishers, Inc.
Pesola, J., Humppi, T., Hakalehto. E. (2015). Method development for Clostridium botulinum toxin detection. In: Hakalehto. E. (ed.) Microbiological Food Hygiene. New York, NY, USA: Nova Science Publishers. Inc.
Schwede, S., Thorin, F., Lindmark, J., Klintenberg, P., Jääskeläinen, A., Suhanen, A., Laatikainen, R., Hakalehto, E (2017). Using slaughterhouse waste in a biochemical based biorefinery result., from pilot scale tests. Environmental Technology, 38: 1275-1284.
Samgina, T. Y., Tolpina. M. I., Hakalehto. E., Artemenko, K. A., Bergquist, J., Lebedev, A. T. (2016). Proteolytic degradation and deactivation of amphibian skin peptides obtained by electrical stimulation of their dorsal glands. Anal. Bioanal. Chem., 408: 3761-3768.

1. A method for microbiological waste treatment, characterized in, that the proteins, fats, and blood fractions in the waste can be dispersed directly in a process reactor with the help of protease, lipases and haemolytic enzymes produced by one or more microbe strains.

2. A method according to patent claim 1, characterized in, that the microbes in question are gram-positive bacteria.

3. A method according to patent claim 2, characterized in, that these bacteria belong to the bacterial genus Staphylococcus.

4. A method according to patent claim 3, characterized in, that the microbes in question belong to the species Staphylococcus haemolyticus.

5. A method according to one or more of patent claims 1-4, characterized in, that the hydrolysed protein, lipid or blood material fractions, that have been separated from other waste material or side streams with the help of microbes, form, when cleansed, usable raw material fractions.

6. A method according to patent claim 5, characterized in, that the waste matter is waste from slaughterhouse or gravy industries.

7. A method according to patent claim 6, characterized in, that bones in the waste can be cleansed with the help of microbes and enzymes produced by them.

8. A method according to one or more of patent claims 1-7, characterized in, that as a selective factor in the growth of bacteria, a suitable salt, and its researched concentration, is used.

9. A method according to patent claim 8, characterized in, that in the selection of Staphylococcus genus bacteria, a NaCl salt concentration of 2.5-7.5% is used.

10. A method according to patent claim 8, characterized in, that in the selection of Staphylococcus bacteria, a NaCl salt concentration of less than 2.5% is used.

11. A method according to one or more of patent claims 1-10, characterized in, that Na ions precipitate fatty acids as a waxlike mass, that can be used as raw material for soap or candles.

12. A method according to one or more of patent claims 1-11, characterized in, that the remaining brine fraction, which may also be a suspension, can be utilized as raw material for a liquid organic fertilizer or biogas.

13. A method according to one or more of patent claims 1-12, characterized in, that the pure culture added to the reactor, is grown on a laboratory medium in a higher salt concentration than what is in the actual inoculation fermenter or the reactor itself.

14. An apparatus for the use of a method according to patent claims 1-13, characterized in, that a reactor can be inoculated with a culture grown in several seed fermenters, which can be added at intervals to the fermenter in question, which seed fermenters are solidly connected to the reactor itself.
